(19) United States
(12) Patent Application Publication
(10) Pub. No.: US 2022/0133216 A1
(43) Pub. Date: May 5, 2022

(54) AUTOMATIC EARLY WARNING METHOD AND SYSTEM FOR PRESSURE SORE PRONE PART OF HUMAN BODY

(71) Applicants: Shenzhen University, Guangdong (CN); Shenzhen University General Hospital, Guangdong (CN)

(72) Inventors: Xiangdong Chen, Guangdong (CN); Huijun Ding, Guangdong (CN); Jing Li, Guangdong (CN)

(21) Appl. No.: 17/431,389

(22) PCT Filed: Jul. 14, 2020
(86) PCT No.: PCT/CN2020/101887

§ 371 Date: Aug. 16, 2021

(30) Foreign Application Priority Data
Jul. 19, 2019 (CN) 201910654175.7

(51) Int. Cl. A61B 5/00

(52) U.S. Cl. CPC A61B 5/447; A61B 5/746; A61B 2562/046; A61B 2562/0247; A61B 5/6802

(57) ABSTRACT

An automatic early warning method and system is for a pressure ulcer prone part of a human body. The method includes: constructing a matrix diagram by using marks with variable states; receiving a pressure signal from a pressure sensor matrix laid in an area that a body of a patient is frequently in contact with; corresponding each miniature ultra-thin dynamic pressure sensor contained in the pressure sensor matrix to each mark in the matrix diagram respectively; after receiving a pressure signal indicating that the pressure at a part in contact with the patient, sensed by the pressure sensor matrix, is greater than or equal to a critical value, changing the state of the marks in the matrix diagram, corresponding to the miniature ultra-thin dynamic pressure sensors in the pressure sensor matrix sensing that the pressure is greater than or equal to the critical value, into an early warning state.

### CROSS-REFERENCE TO RELATED APPLICATION

This application is the U.S. National Stage of PCT/CN2020/101887 filed Jul. 14, 2020, which claims priority to Chinese Patent Application No. 201910654175.7, filed Jul. 19, 2019, the content of both are incorporated herein by reference in their entirety.

### FIELD OF THE INVENTION

The present application relates to the field of anthropometric technology, and in particular to an automatic early warning method and system for a pressure ulcer prone part of human body.

### BACKGROUND OF THE INVENTION

Pressure ulcer, also known as pressure injury, pressure sore and bedsore, is caused by long-term pressure on local tissues, resulting in continuous ischemia, hypoxia, and malnutrition, which can cause tissue ulceration and necrosis, and the pressure ulcer is generally a complication of the patient who cannot easily or cannot move.

The formation of the pressure ulcer is a process, and before the pressure ulcer is formed, identify the pressure ulcer prone parts on the body of the patient, and perform decompression, massage and other nursing on the pressure ulcer prone parts to reduce the occurrence of pressure ulcers. At present, determining the pressure ulcer prone parts is to regularly observe whether the skin of the pressed parts of the patient has changes in color, such as the skin turning purple or brownish red, or it is accompanied by pain, induration, fever, or coldness at the same time.

However, since each person has a different body size and weight, different people have different pressure ulcer prone parts at different positions of the body, it is necessary for the nursing staff to have a rich pressure ulcer management experience to determine whether the local skin on the body of the patient is a pressure ulcer prone part.

### SUMMARY OF THE INVENTION

The main objective of the present application is to provide an automatic early warning method and system for a pressure ulcer prone part of human body, to solve the technical problem that it is necessary for the nursing staff to have a rich pressure ulcer management experience to determine whether the local skin on the body of the patient is a pressure ulcer prone part in the prior art.

In order to achieve the above purpose, a first aspect of the present application provides an automatic early warning method for a pressure ulcer prone part of human body, comprising: constructing a matrix diagram by using marks with variable states; receiving a pressure signal from a pressure sensor matrix laid in an area that a body of a patient is frequently in contact with; corresponding each miniature ultra-thin dynamic pressure sensor contained in the pressure sensor matrix to each mark in the matrix diagram respectively; after receiving a pressure signal indicating that the pressure at a part in contact with the patient, sensed by the pressure sensor matrix, is greater than or equal to a critical value, changing the state of the marks in the matrix diagram, corresponding to the miniature ultra-thin dynamic pressure sensors in the pressure sensor matrix sensing that the pressure is greater than or equal to the critical value, into an early warning state.

Preferably, the method further comprises: when the state of the marks becomes the early warning state and after the early warning state lasts for a preset duration, sending out a sound warning.

Preferably, the critical value is 32 mmHg, and the preset duration is 6 minutes.

Preferably, the method further comprises: when the state of the marks is a non-early warning state, using different marks to represent different pressures sensed by different miniature ultra-thin dynamic pressure sensors in the pressure sensor matrix.

Preferably, constructing a matrix diagram by using marks with variable states comprises using mark points with different colors as marks, and using the mark points with different colors as marks for different states to construct a matrix diagram, or using different numerical values as marks, and using the different numerical values as the marks for different states to construct the matrix diagram.

Preferably, the method further comprises receiving a data signal from wearable equipment, composed of miniature ultra-thin dynamic pressure sensors, worn by the patient; constructing a corresponding marking map according to spatial distribution of the miniature ultra-thin dynamic pressure sensors on the wearable equipment.

A second aspect of the present application provides an automatic early warning system for a pressure ulcer prone part of human body, comprising: a sensor matrix module, configured to sense a pressure at a part that a patient is in contact with and generating a pressure signal; a matrix diagram constructing module, configured to construct a matrix diagram by using marks with variable states; a pressure signal receiving module, configured to receive the pressure signal generated by the sensor matrix module; a corresponding module, configured to correspond each miniature ultra-thin dynamic pressure sensor contained in the sensor matrix module to each mark in the matrix diagram constructed by the matrix diagram constructing module respectively; an early warning module, configured to change the state of the marks in the matrix diagram in the matrix diagram constructing module, corresponding to the miniature ultra-thin dynamic pressure sensors in the pressure sensor matrix sensing that the pressure is greater than or equal to the critical value, into an early warning state, after the pressure signal receiving module receives the pressure signal indicating that the pressure at a part in contact with the patient, sensed by the sensor matrix module, is greater than or equal to a critical value.

Preferably, the system further comprises a warning module, configured to send out a sound warning after the early warning module changes the state of the marks of the matrix diagram in the matrix diagram constructing module to an early warning state and the early warning state lasts for a preset duration.

The present application provides an automatic early warning method and system for a pressure ulcer prone part of human body, and the beneficial effect is that: through using the pressure sensor matrix, it can be accurately known whether the pressure at the part where the patient contacts the pressure sensor matrix reaches the critical value, so that the pressure on the surface of different parts of the body of the patient can be detected automatically, the nursing staff only need to observe whether the mark is in the early warning state to determine whether the local skin of the body of the patient is the pressure ulcer prone part, thereby reducing the requirements for the pressure ulcer management experience of the nursing staff.

### BRIEF DESCRIPTION OF THE DRAWINGS

In order to more clearly illustrate the embodiments of the present application or the technical solutions in the prior art, the accompanying drawings used in describing the embodiments or the prior art will be briefly introduced below, and apparently, the accompanying drawings mentioned in the following description are just some embodiments of the present application, and other drawings can be derived by a person skilled in the art from these drawings without expenditure of creative efforts.

FIG. 1 is a flow schematic block diagram of an automatic early warning method for a pressure ulcer prone part of human body in an embodiment of the present application;

FIG. 2 is a structure schematic block diagram of an automatic early warning system for a pressure ulcer prone part of human body in an embodiment of the present application.

### DETAILED DESCRIPTION OF EMBODIMENTS

In order to enable the purpose, features and advantages of the present application to be more obvious and understandable, the technical solutions in the embodiments of the present application will be clearly and completely described below in conjunction with the accompanying drawings in the embodiments of the present application Apparently, the described embodiments are only a part of, but not all of, the embodiments of the present application. All other embodiments obtained by a person skilled in the art based on the embodiments described in the present application without expenditure of creative efforts belong to the protection scope of the present application.

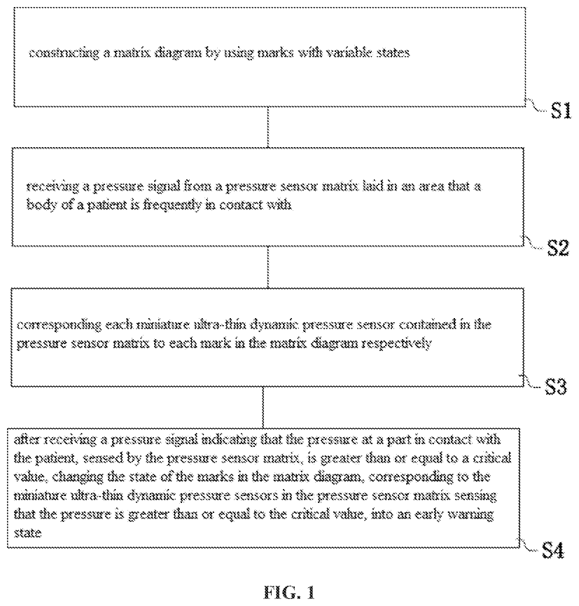

Referring to FIG. 1, which is automatic early warning method for a pressure ulcer prone part of human body, comprising: S1, constructing a matrix diagram by using marks with variable states; S2, receiving a pressure signal from a pressure sensor matrix laid in an area that a body of a patient is frequently in contact with; S3, corresponding each miniature ultra-thin dynamic pressure sensor contained in the pressure sensor matrix to each mark in the matrix diagram respectively; S4, after receiving a pressure signal indicating that the pressure at a part in contact with the patient, sensed by the pressure sensor matrix, is greater than or equal to a critical value, changing the state of the marks in the matrix diagram, corresponding to the miniature ultra-thin dynamic pressure sensors in the pressure sensor matrix sensing that the pressure is greater than or equal to the critical value, into an early warning state.

In the present embodiment, the area of the pressure sensor matrix is 190 cm x90 cm, which is suitable for detecting the lying position of a patient with a common size, and each miniature ultra-thin dynamic pressure sensor is embedded in each 2 cm x2 cm (4 cm²) of the pressure sensor matrix, and 4275 sensors are provided. The pressure information sensed by 4275 miniature ultra-thin dynamic pressure sensors is processed by an integrated pressure signal processing system, and the pressure information of different parts of the patient is displayed on a computer screen in the form of a pressure distribution diagram, and the pressure distribution diagram is superposed with the matrix diagram.

The automatic early warning method for a pressure ulcer prone part of human body further comprises: when the state of the marks becomes the early warning state and after the early warning state lasts for a preset duration, sending out a sound warning.

In the present embodiment, the critical value is 32 mmHg, and the preset duration is 6 minutes.

Generally, the decompression nursing and the massage nursing are performed on the severely pressed part of the body of the patient every 6 minutes, and when the nursing staff does not find that the mark is changed into the early warning state for 6 minutes, a sound warning is sent out to remind the nursing staff, so that the possibility of occurrence of the incident that the nursing staff inaccurately controls the duration is reduced.

The automatic early warning method for a pressure ulcer prone part of human body further comprises: when the state of the marks is a non-early warning state, using different marks to represent different pressures sensed by different miniature ultra-thin dynamic pressure sensors in the pressure sensor matrix. Constructing a matrix diagram by using marks with variable states comprises: using mark points with different colors as marks, and using the mark points with different colors as marks for different states to construct a matrix diagram, or using different numerical values as marks, and using the different numerical values as the marks for different states to construct the matrix diagram.

In the present embodiment, the marks are formed by mark points with different colors, when the marks are in the early warning state, the mark points are black and flashing so as to attract the attention of the nursing staff; in other embodiments, the marks are formed by different values, and the values are used for directly reflecting the pressure values sensed by the miniature ultra-thin dynamic pressure sensors, so that the pressure values of the pressed parts of the patient are more visual and clear.

It can be known that, when a human body is on the sensor matrix, not all ultra-thin dynamic pressure sensors on the sensor matrix are subjected to pressure, therefore, in the present embodiment, different pressures on different parts of the human body to the sensor matrix are displayed in different states on the corresponding matrix diagram, so that the matrix diagram presents a human body outline, in the present embodiment, the ultra-thin dynamic pressure sensors that are not subjected to pressure of the human body present white mark points on the matrix diagram, the ultra-thin dynamic pressure sensors that are subjected to slight pressure of the human body present green mark points on the matrix diagram, and when the pressure on the ultra-thin dynamic pressure sensor exceeds 32 mmHg, the corresponding mark point on the matrix diagram is black and flashing; in other embodiments, other colors can be used to represent the colors of the ultra-thin dynamic pressure sensors on the matrix diagram after the ultra-thin dynamic pressure sensors are subjected to the pressure of the human body.

The automatic early warning method for a pressure ulcer prone part of human body further comprises receiving a data signal from wearable equipment, composed of miniature ultra-thin dynamic pressure sensors, worn by the patient, and constructing a corresponding marking map according to spatial distribution of the miniature ultra-thin dynamic pressure sensors on the wearable equipment.

The use of wearable equipment can meet the detection needs of the different body positions and different parts of the body of patient, thus making the pressure monitoring of patients more comprehensive, therefore, reducing the risk of patients with pressure ulcers.

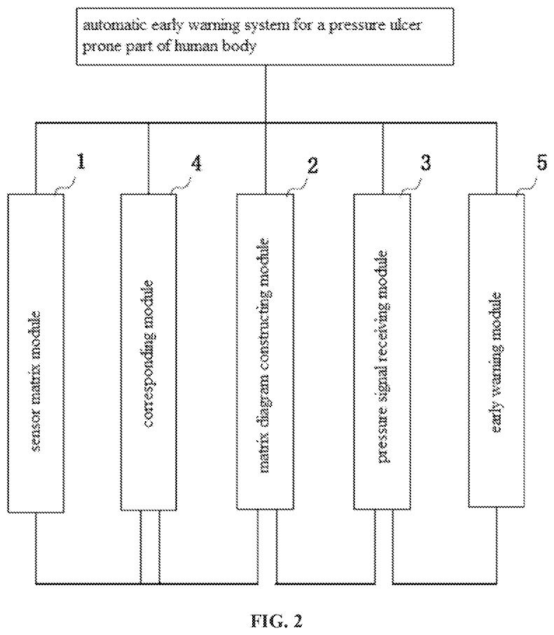

Referring to FIG. 2, which is an automatic early warning system for a pressure ulcer prone part of human body, comprising: the device comprises a sensor matrix module 1, a matrix diagram constructing module 2, a pressure signal receiving module 3, a corresponding module 4 and an early warning module 5; the sensor matrix module 1 is configured to sense a pressure at a part that a patient is in contact with and generating a pressure signal; the matrix diagram constructing module 2 is configured to construct a matrix diagram by using marks with variable states; the pressure signal receiving module 3 is configured to receive the pressure signal generated by the sensor matrix module 1; the corresponding module 4 is configured to correspond each miniature ultra-thin dynamic pressure sensor contained in the sensor matrix module 1 to each mark in the matrix diagram constructed by the matrix diagram constructing module 2 respectively; the early warning module 5 is configured to change the state of the marks in the matrix diagram in the matrix diagram constructing module 2, corresponding to the miniature ultra-thin dynamic pressure sensors in the pressure sensor matrix sensing that the pressure is greater than or equal to the critical value, into an early warning state, after the pressure signal receiving module 3 receives the pressure signal indicating that the pressure at a part in contact with the patient, sensed by the sensor matrix module 1, is greater than or equal to a critical value.

The system further comprises a warning module, which is configured to send out a sound warning after the early warning module 5 changes the state of the marks of the matrix diagram in the matrix diagram constructing module 2 to an early warning state and the early warning state lasts for a preset duration.

The system further comprises a threshold setting module, which is configured to set a critical value to change the state of the marks in the matrix diagram in the matrix diagram constructing module 2, corresponding to the miniature ultra-thin dynamic pressure sensors in the pressure sensor matrix sensing that the pressure is greater than or equal to the critical value, into an early warning state, after the pressure signal receiving module 3 receives the pressure signal indicating that the pressure at a part in contact with the patient, sensed by the sensor matrix module 1, is greater than or equal to a critical value. the threshold setting module is also configured to set a preset duration to send out a sound warning after the early warning module 5 changes the state of the marks of the matrix diagram in the matrix diagram constructing module 2 to an early warning state and the early warning state lasts for a preset duration.

The system further comprises a pressure representation module, which is configured to use different marks to represent different pressures sensed by different miniature ultra-thin dynamic pressure sensors in the sensor matrix module 1 when the marks of the matrix diagram in the matrix diagram constructing module 2 is in the non-early warning state.

In the present embodiment, the matrix diagram constructing module 2 uses mark points with different colors as marks, and using the mark points with different colors as marks for different states to construct a matrix diagram, and in other embodiments, uses different numerical values as marks, and using the different numerical values as the marks for different states to construct the matrix diagram.

The system further comprises a data signal receiving module and a marking map constructing module; wherein the data signal receiving module is configured to receive a data signal from wearable equipment, composed of miniature ultra-thin dynamic pressure sensors, worn by the patient; and the marking map constructing module is configured to construct a corresponding marking map according to spatial distribution of the miniature ultra-thin dynamic pressure sensors on the wearable equipment.

The automatic early warning method for a pressure ulcer prone part of human body provided by the present application is described above. For those skilled in the art, according to the ideas of the embodiments of the present application, there will be changes in the specific implementation and the scope of application. In summary, the content of the present specification should not be construed as a limitation on the present application.

1. An automatic early warning method for a pressure ulcer prone part of a human body, comprising:
constructing a matrix diagram by using marks with variable states;
receiving a pressure signal from a pressure sensor matrix laid in an area that a body of a patient is frequently in contact with;
corresponding each miniature ultra-thin dynamic pressure sensor contained in the pressure sensor matrix to each mark in the matrix diagram respectively;
after receiving a pressure signal indicating that the pressure at a part in contact with the patient, sensed by the pressure sensor matrix, is greater than or equal to a critical value, changing the state of the marks in the matrix diagram, corresponding to the miniature ultra-thin dynamic pressure sensors in the pressure sensor matrix sensing that the pressure is greater than or equal to the critical value, into an early warning state.

2. The automatic early warning method for a pressure ulcer prone part of a human body according to claim 1, further comprising:
when the state of the marks becomes the early warning state and after the early warning state lasts for a preset duration, sending out a sound warning.

3. The automatic early warning method for a pressure ulcer prone part of a human body according to claim 2, wherein, the critical value is 32 mmHg, and the preset duration is 6 minutes.

4. The automatic early warning method for a pressure ulcer prone part of a human body according to claim 1, further comprising:
when the state of the marks is a non-early warning state, using different marks to represent different pressures sensed by different miniature ultra-thin dynamic pressure sensors in the pressure sensor matrix.

5. The automatic early warning method for a pressure ulcer prone part of a human body according to claim 1, wherein:
the step of constructing a matrix diagram by using marks with variable states comprises:
using mark points with different colors as marks, and using the mark points with different colors as marks for different states to construct a matrix diagram; or
using different numerical values as marks, and using the different numerical values as the marks for different states to construct the matrix diagram.

6. The automatic early warning method for a pressure ulcer prone part of a human body according to claim 1, further comprising:
receiving a data signal from wearable equipment, composed of miniature ultra-thin dynamic pressure sensors, worn by the patient;
constructing a corresponding marking map according to a spatial distribution of the miniature ultra-thin dynamic pressure sensors on the wearable equipment.

7. An automatic early warning system for a pressure ulcer prone part of a human body, comprising:
a sensor matrix module, configured to sense a pressure at a part that a patient is in contact with and generating a pressure signal;
a matrix diagram constructing module, configured to construct a matrix diagram by using marks with variable states;
a pressure signal receiving module, configured to receive the pressure signal generated by the sensor matrix module;
a corresponding module, configured to correspond each miniature ultra-thin dynamic pressure sensor contained in the sensor matrix module to each mark in the matrix diagram constructed by the matrix diagram constructing module respectively; and
an early warning module, configured to change the state of the marks in the matrix diagram in the matrix diagram constructing module, corresponding to the miniature ultra-thin dynamic pressure sensors in the pressure sensor matrix sensing that the pressure is greater than or equal to the critical value, into an early warning state, after the pressure signal receiving module receives the pressure signal indicating that the pressure at a part in contact with the patient, sensed by the sensor matrix module, is greater than or equal to a critical value.

8. The automatic early warning system for a pressure ulcer prone part of a human body according to claim 7, further comprising:
a warning module, configured to send out a sound warning after the early warning module changes the state of the marks of the matrix diagram in the matrix diagram constructing module to an early warning state and the early warning state lasts for a preset duration.
